(19) United States
(12) Patent Application Publication
(10) Pub. No.: US 2024/0423444 A1
(43) Pub. Date: Dec. 26, 2024

(54) PROCESSING DEVICE, ENDOSCOPE DEVICE, PROCESSING METHOD, AND PROCESSING PROGRAM

(71) Applicant: FUJIFILM Corporation, Tokyo (JP)

(72) Inventor: Masaaki OOSAKE, Ashigarakami-gun (JP)

(73) Assignee: FUJIFILM Corporation, Tokyo (JP)

(21) Appl. No.: 18/747,456

(22) Filed: Jun. 19, 2024

(30) Foreign Application Priority Data
Jun. 21, 2023 (JP) 2023-101417

(51) Int. Cl. A61B 1/00; G06T 7/60

(52) U.S. Cl. CPC A61B 1/00009; A61B 1/00158; G06T 7/60; G06T 2207/10068; G06T 2207/30004

(57) ABSTRACT

A processing device includes a processor. The processor is configured to: acquire first medical image data and second medical image data that include a specific region in a subject as medical image data acquired by a medical image acquisition device; acquire first information regarding an imaging position of the first medical image data and second information regarding an imaging position of the second medical image data; and derive an actual size of the specific region based on the first medical image data, the second medical image data, the first information, and the second information.

### CROSS-REFERENCE RELATED APPLICATIONS

This application is based on and claims priority under 35 USC 119 from Japanese Patent Application No. 2023-101417 filed on Jun. 21, 2023, the contents of which are incorporated herein by reference.

### BACKGROUND OF THE INVENTION

### 1. Field of the Invention

The present invention relates to a processing device, an endoscope device, a processing method, and a computer-readable storage medium storing a processing program.

### 2. Description of the Related Art

International Patent Application Publication No. 2018-180250 discloses an endoscope system comprising a measurement mode of generating and displaying a marker for measuring a size of a subject based on an image of the subject on which a spot is formed by emitting auxiliary measurement light to the subject.

### SUMMARY OF THE INVENTION

In the present disclosure, it is possible to provide a technique capable of deriving the size of a specific region in a subject to be examined without increasing a cost.

According to one aspect of the present disclosure, there is provided a processing device comprising: a processor, in which the processor is configured to: acquire first medical image data and second medical image data including a specific region in a subject as medical image data acquired by a medical image acquisition device; acquire first information regarding an imaging position of the first medical image data and second information regarding an imaging position of the second medical image data; and derive an actual size of the specific region based on the first medical image data, the second medical image data, the first information, and the second information.

An endoscope device according to another aspect of the present disclosure includes the processing device and the endoscope.

According to an aspect of the present disclosure, there is provided a processing method comprising: acquiring first medical image data and second medical image data including a specific region in a subject as medical image data acquired by a medical image acquisition device; acquiring first information regarding an imaging position of the first medical image data and second information regarding an imaging position of the second medical image data; and deriving an actual size of the specific region based on the first medical image data, the second medical image data, the first information, and the second information.

According to one aspect of the present disclosure, there is provided a non-transitory computer-readable storage medium storing a processing program that causes a processor to execute process including: acquiring first medical image data and second medical image data including a specific region in a subject as medical image data acquired by a medical image acquisition device; acquiring first information regarding an imaging position of the first medical image data and second information regarding an imaging position of the second medical image data; and deriving an actual size of the specific region based on the first medical image data, the second medical image data, the first information, and the second information.

According to the present disclosure, it is possible to provide a technique capable of deriving the size of a specific region in a subject to be examined without increasing a cost.

### BRIEF DESCRIPTION OF THE DRAWINGS

FIG. 1 is a diagram showing a schematic configuration of an endoscope system 200.

FIG. 2 is a partial cross-sectional view showing a detailed configuration of a soft portion 10A of an endoscope 1.

FIG. 3 is a schematic diagram showing details of a magnetic pattern formed on a tubular member 17.

FIG. 4 is a schematic cross-sectional view taken along each of an A-A arrow and a B-B arrow in FIG. 3.

FIG. 5 is an exploded perspective view showing a configuration example of a detection unit 40.

FIG. 6 is a schematic diagram of a body part 42A of the detection unit 40 shown in FIG. 5 as viewed from a direction x.

FIG. 7 is a diagram showing an example of a position at which an insertion part 10 can be positioned in a through-hole 41.

FIG. 8 is a schematic diagram showing an example of a magnetic flux density detected by a magnetic detection unit 43.

FIG. 9 is a schematic diagram showing an example of a result of classifying the magnetic flux density shown in FIG. 8 according to magnitude thereof.

FIG. 10 is a schematic diagram showing another example of the magnetic flux density shown in FIG. 8 according to the magnitude thereof.

FIG. 11 is a schematic diagram for illustrating a movement path of insertion part 10 in examination performed using the endoscope 1.

FIG. 12 is a diagram showing a transition example of a screen displayed on the display device 7.

### DESCRIPTION OF THE PREFERRED EMBODIMENTS

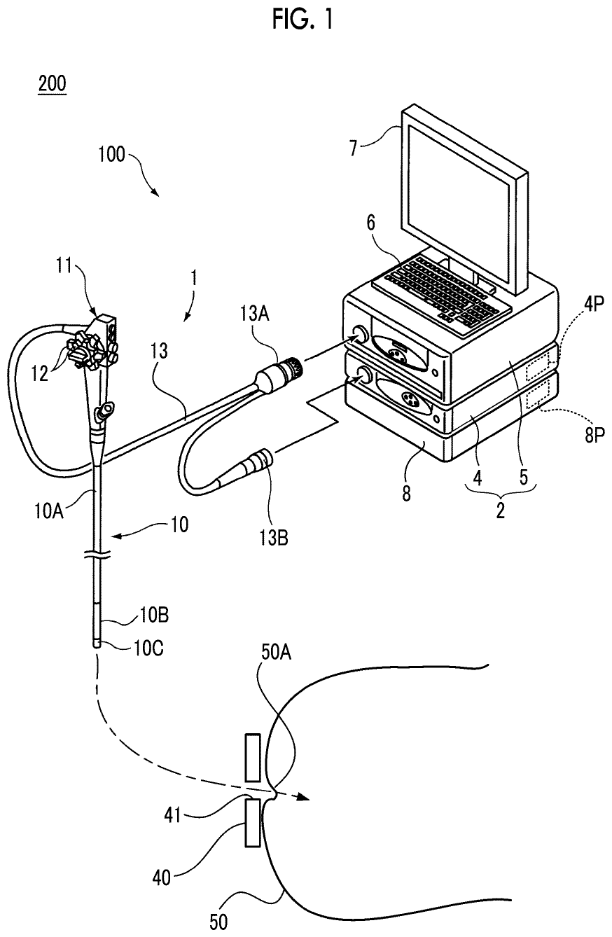

FIG. 1 is a diagram showing a schematic configuration of an endoscope system 200. The endoscope system 200 includes an endoscope device 100 having an endoscope 1 as an example of a medical image acquisition device that is used by being inserted into a body for examination, surgery, and the like, and a detection unit 40.

The endoscope 1 includes an insertion part 10 which is an elongated instrument extending in one direction and is inserted into the body, an operating part 11 which is provided on a proximal end of the insertion part 10 and is provided with an operation member for performing an observation mode switching operation, an imaging recording operation, a forceps operation, an air supply and water supply operation, a suction operation, an electric cautery operation, or the like, an angle knob 12 provided adjacent to the operating part 11, and a universal cord 13 including connector portions 13A and 13B that respectively connect the endoscope 1 to a light source device 5 and a processor device 4 in an attachable and detachable manner.

The operating part 11 is provided with a forceps port into which biopsy forceps as a treatment tool for collecting a biological tissue such as a cell or a polyp are inserted. It should be noted that, although the illustration is omitted in FIG. 1, various channels such as a forceps channel through which the biopsy forceps inserted from the forceps port are inserted, a channel for air supply and water supply, and a channel for suction are provided inside the operating part 11 and the insertion part 10.

The insertion part 10 includes a soft portion 10A having flexibility, a bendable part 10B provided at a distal end of the soft portion 10A, and a distal end part 10C that is provided at a distal end of the bendable part 10B and that is harder than the soft portion 10A. An imaging element and an imaging optical system are built in the distal end part 10C.

The bendable part 10B is configured to be bendable by a rotational movement operation of the angle knob 12. Depending on a site or the like of a subject in which the endoscope 1 is used, the bendable part 10B can be bent in any direction and at any angle, and the distal end part 10C can be directed in a desired direction.

Hereinafter, a direction in which the insertion part 10 extends will be referred to as a longitudinal direction X. Further, one of radial directions of the insertion part 10 will be referred to as a radial direction Y. In addition, one of circumferential directions of the insertion part 10 (one of tangential directions of an outer peripheral edge of the insertion part 10) will be referred to as a circumferential direction Z. In the longitudinal direction X, a direction from a base end (operating part 11 side) of the endoscope 1 toward a distal end will be referred to as a longitudinal direction X1, and a direction from the distal end of the endoscope 1 to the base end will be referred to as a longitudinal direction X2. In addition, in the radial direction Y, one side will be referred to as a radial direction Y1, and the other side will be referred to as a radial direction Y2. The longitudinal direction X is one of directions different from the radial direction Y and the circumferential direction Z. The radial direction Y is one of directions different from the longitudinal direction X and the circumferential direction Z. In the present specification, the longitudinal direction X constitutes a first direction. Further, the radial direction Y constitutes a second direction intersecting the first direction. Further, the circumferential direction Z constitutes a third direction different from the first direction and the second direction.

In the example of FIG. 1, the insertion part 10 of the endoscope 1 is inserted into the body of a subject 50 from an anus 50A of the subject 50. The detection unit 40 has a rectangular plate shape as an example, and has a through-hole 41 into which the insertion part 10 can be inserted. The detection unit 40 is disposed between buttocks of the subject 50 and the insertion part 10 (that is, a movement path of the insertion part 10). The insertion part 10 reaches the anus 50A through the through-hole 41 of the detection unit 40, and is inserted into the body of the subject 50 from the anus 50A. In the present specification, the insertion part 10 constitutes an elongated instrument that is used by being relatively moved with respect to the detection unit 40.

The endoscope device 100 includes the endoscope 1, a body part 2 consisting of the processor device 4 and the light source device 5 to which the endoscope 1 is connected, a display device 7 that displays a captured image and the like, an input unit 6 that is an interface for inputting various kinds of information to the processor device 4, and an expansion device 8 for expanding various functions.

The processor device 4 has various processors 4P that control the endoscope 1, the light source device 5, and the display device 7. The expansion device 8 has a processor 8P that performs various kinds of processing. Each of the processor 4P and the processor 8P is a central processing unit (CPU) as a general-purpose processor that executes software (a program including a processing program) to perform various functions, a programmable logic device (PLD) as a processor of which a circuit configuration can be changed after the manufacture, such as a field programmable gate array (FPGA), and a dedicated electric circuit as a processor having a circuit configuration specially designed for executing specific processing, such as an application specific integrated circuit (ASIC). Each of the processor 4P and the processor 8P may be composed of one processor, or composed of a combination of two or more processors of the same type or a different type (for example, a combination of a plurality of FPGAs or a combination of a CPU and an FPGA). More specifically, the hardware structure of each of the processor 4P and the processor 8P is an electric circuit (circuitry) in which circuit elements such as semiconductor elements are combined.

The expansion device 8 includes the processor 8P, a communication interface (an interface for communicating with the processor device 4 and the detection unit 40 to be described later) (not shown), and a memory composed of a recording medium such as a random access memory (RAM), a read only memory (ROM), a solid state drive (SSD), or a hard disk drive (HDD), and constitutes a processing device.

The processor 8P may acquire the captured image data captured by the endoscope 1 from the processor device 4 and perform lesion recognition processing of recognizing a lesion region in the captured image data, treatment tool recognition processing of recognizing whether or not a treatment tool such as a forceps or a needle is included in the captured image data, and the like.

The lesion recognition processing refers to processing for performing detection of the lesion region from the captured image data, and identification of the detected lesion region. In the lesion recognition processing, the processing for detecting the lesion region is referred to as detection processing, and the processing for identifying the lesion region is referred to as identification processing. The lesion recognition processing may be processing including at least the detection processing. The detection of the lesion region refers to finding a lesion region suspected of a lesion such as a malignant tumor or a benign tumor (lesion candidate region), from the captured image. The identification of the lesion region refers to identifying the type, nature, and the like of the detected lesion region, such as whether the lesion region detected by the detection processing is malignant or benign, and if it is malignant, what kind of disease it is or how advanced the disease is. For example, both the lesion recognition processing and the treatment tool recognition processing can be executed by a recognition model generated by machine learning (for example, a neural network or a support vector machine) or image analysis on the captured image data. The lesion candidate region constitutes the specific region.

The various kinds of processing described below performed by the processor 8P may be performed by the processor 8P alone, or may be performed by being shared between the processor 8P and another processor. The other processor is, for example, a processor of a server in an examination system in which examination data generated by the endoscope system 200 is recorded, the processor 4P, or the like. Alternatively, various kinds of processing performed by the processor 8P can be performed by the processor 4P.

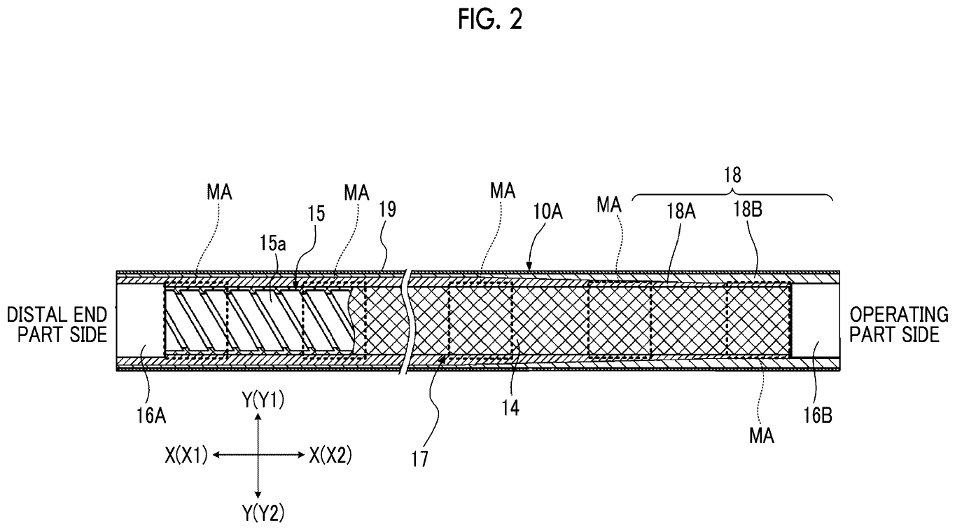

FIG. 2 is a partial cross-sectional view showing a detailed configuration of the soft portion 10A of the endoscope 1. The soft portion 10A, which forms most of a length of the insertion part 10, has flexibility over substantially the entire length thereof, and has a structure in which, in particular, a portion to be inserted into a body cavity or the like is highly flexible.

The soft portion 10A includes an outer skin layer 18 that constitutes a cylindrical member having an insulating property, and a tubular member 17 that is provided in the outer skin layer 18. The outer skin layer 18 is coated with a coating layer 19.

The tubular member 17 includes a first member 14 that has a cylindrical shape, contains metal, and is covered with the outer skin layer 18; and a second member 15 that has a cylindrical shape, contains metal, and is inserted into the first member 14. In the example of FIG. 2, the second member 15 is composed of a spiral tube formed by spirally winding a metal strip 15a. Further, the first member 14 is composed of a cylindrical-shaped net body formed by braiding a metal wire. The first member 14 and the second member 15 that continuously extend in the longitudinal direction X and have a thin structure are formed by plastic processing, and the metal constituting these members includes austenitic stainless steel. The austenitic stainless steel cannot be magnetized in a state in which the plastic processing is not performed, but can be magnetized by performing the plastic processing. As described above, each of the first member 14 and the second member 15 constitutes a member that extends in the longitudinal direction X and that contains metal.

The outer skin layer 18 is composed of, for example, a resin such as an elastomer, and has a multi-layer structure of an inner resin layer 18A and an outer resin layer 18B. The outer skin layer 18 may have a monolayer structure. In the first member 14 and the second member 15, a cap 16A is fitted to an end part on the distal end part 10C side, and a cap 16B is fitted to an end part on the operating part 11 side. The cap 16A and the cap 16B are covered with the outer skin layer 18. The soft portion 10A is connected to the bendable part 10B at the cap 16A, and is connected to the operating part 11 at the cap 16B.

The tubular member 17 of the soft portion 10A is formed with a magnetic pattern along the longitudinal direction X. The magnetic pattern along the longitudinal direction X refers to a pattern in which two types of magnetic pole regions, which are a negative pole (S pole) and a positive pole (N pole), are arranged in a predetermined arrangement pattern in the longitudinal direction X. As shown in FIG. 2, each of the first member 14 and the second member 15 is provided with a plurality of magnetic pole portions MA including the magnetic pole region. At least one of the two types of magnetic pole regions, which are the negative pole (S pole) and the positive pole (N pole), is formed on the magnetic pole portion MA. As described above, each of the first member 14 and the second member 15 constitutes the member that extends in the longitudinal direction X and that has the magnetic pattern formed along the longitudinal direction X.

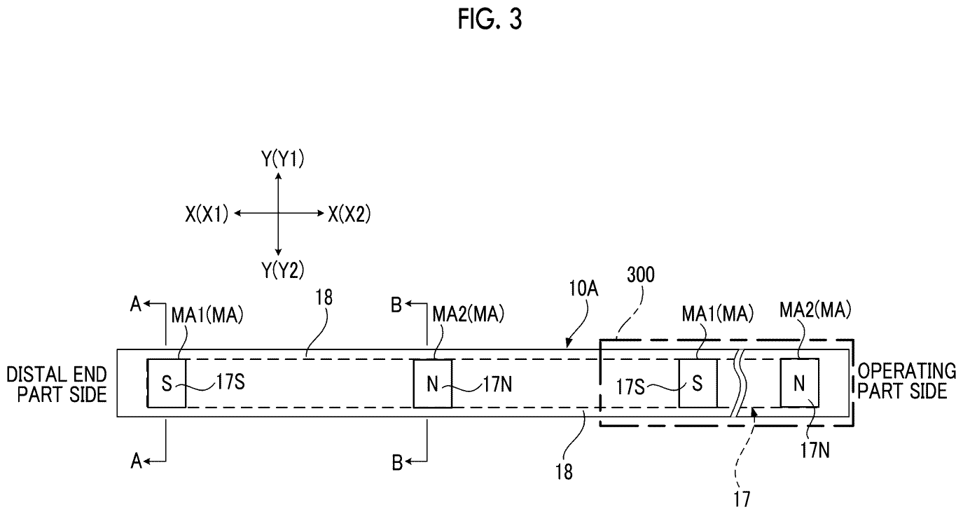

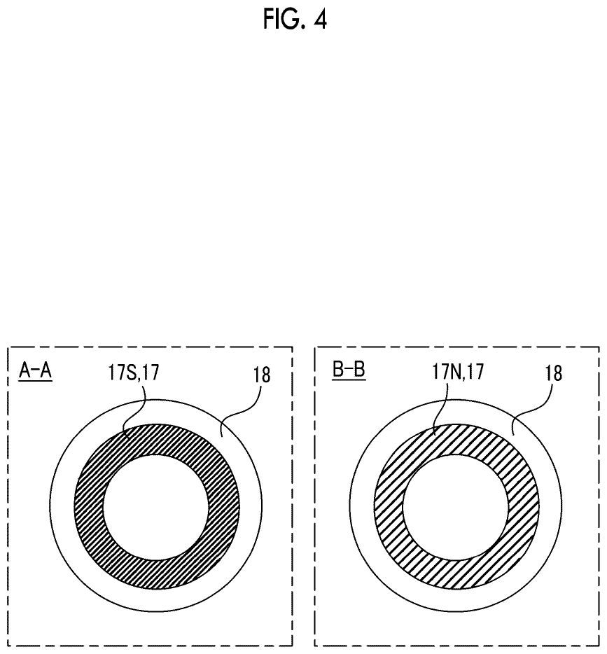

FIG. 3 is a schematic diagram showing details of the magnetic pattern formed on the tubular member 17. FIG. 4 is a schematic cross-sectional view taken along each of an A-A arrow and a B-B arrow in FIG. 3. As shown in FIGS. 3 and 4, in the tubular member 17, a magnetic pole portion MA1 including a negative pole region 17S formed in an annular shape along the circumferential direction of the tubular member 17, and a magnetic pole portion MA2 including a positive pole region 17N formed in an annular shape along the circumferential direction of the tubular member 17 are provided to be alternately arranged in the longitudinal direction X. The total number of the magnetic pole portions MA1 and the total number of the magnetic pole portions MA2 are the same.

Here, an example of a manufacturing method of the endoscope 1 including the tubular member 17 having the magnetic pattern shown in FIG. 3 will be described. First, the endoscope 1 having the configuration shown in FIG. 1 is manufactured by a well-known method. Next, a magnetic field generation device 300 is prepared, which has a cylindrical coil, and can generate a magnetic field in the cylindrical coil by allowing a current to flow through the cylindrical coil. Next, as shown in FIG. 3, the insertion part 10 of the endoscope 1 is inserted into the cylindrical coil of the magnetic field generation device 300 from the distal end side to relatively move the coil to a boundary portion between the operating part 11 and the soft portion 10A. In this state, a step of allowing an alternating current to flow through the cylindrical coil of the magnetic field generation device 300 to form a magnetic field, and pulling out the insertion part 10 from the cylindrical coil of the magnetic field generation device 300 in the longitudinal direction X2 at a constant speed is performed. In this step, a magnetic force of the tubular member 17 generated by the plastic processing is removed, and the tubular member 17 is demagnetized. In this step, it is preferable to pull out the insertion part 10 until the bendable part 10B and the distal end part 10C pass through the cylindrical coil, and to demagnetize the entire insertion part 10. That is, in the insertion part 10 of the endoscope 1, it is preferable that the bendable part 10B and the distal end part 10C are demagnetized. The demagnetization of a certain region means that a magnetic flux density detected from the region is equal to or less than the geomagnetism.

After the demagnetization of at least the tubular member 17 (soft portion 10A) is performed, work of forming a state in which the cylindrical coil of the magnetic field generation device 300 is disposed on an outer periphery of the soft portion 10A at a predetermined position in the longitudinal direction X, and of allowing the alternating current to flow through the cylindrical coil in that state to form the magnetic field is performed. By this work, the negative pole region 17S and the positive pole region 17N are formed over the entire circumferential direction of the tubular member 17 at positions in the vicinity of both ends of the cylindrical coil of the magnetic field generation device 300. Thereafter, by repeating this work while shifting the position of the soft portion 10A with respect to the cylindrical coil in the longitudinal direction X, the magnetic pattern shown in FIG. 3 can be formed on the tubular member 17.

By adopting such a manufacturing method, any magnetic pattern can be easily formed on the tubular member 17 of the soft portion 10A even in the endoscope 1 having the existing configuration or the endoscope 1 that has already been sold. In addition, by performing the demagnetization of the tubular member 17 of the soft portion 10A and then forming the magnetic pattern on the tubular member 17, the magnetic pattern having a desired magnetic force can be formed with high accuracy. Further, by forming the magnetic pole region by using the cylindrical coil, it is possible to form the magnetic pole region having a uniform magnetic force (magnetic flux density) over the entire outer periphery of the tubular member 17 in the magnetic pole portion MA. In FIG. 3, a boundary line between each of the negative pole region 17S and the positive pole region 17N, and the other region in the tubular member 17 is shown, but this boundary line is shown for convenience, and is invisible. It is preferable that information on the magnetic pattern formed on the tubular member 17 is recorded in a memory (for example, a memory provided in the expansion device 8) accessible by the processor 8P. The information on the magnetic pattern includes information indicating positions of the two types of magnetic pole regions in the tubular member 17, information indicating an arrangement pitch of the two types of magnetic pole regions in the tubular member 17, information indicating a range in which the magnetic pole region is formed on the insertion part 10, information indicating the position of the demagnetized region in the insertion part 10, and the like. The demagnetized region in the insertion part 10 constitutes an adjacent region adjacent to the region in which the magnetic pattern is formed in the insertion part 10. The bendable part 10B and the distal end part 10C are demagnetized regions in the insertion part 10, but the bendable part 10B and the distal end part 10C need only be configured to be distinguishable from the region in which the magnetic pattern is formed, and it is not essential that the bendable part 10B and the distal end part 10C are demagnetized. For example, magnetization may be performed with a pattern or a magnetic force that is clearly different from the magnetic pattern.

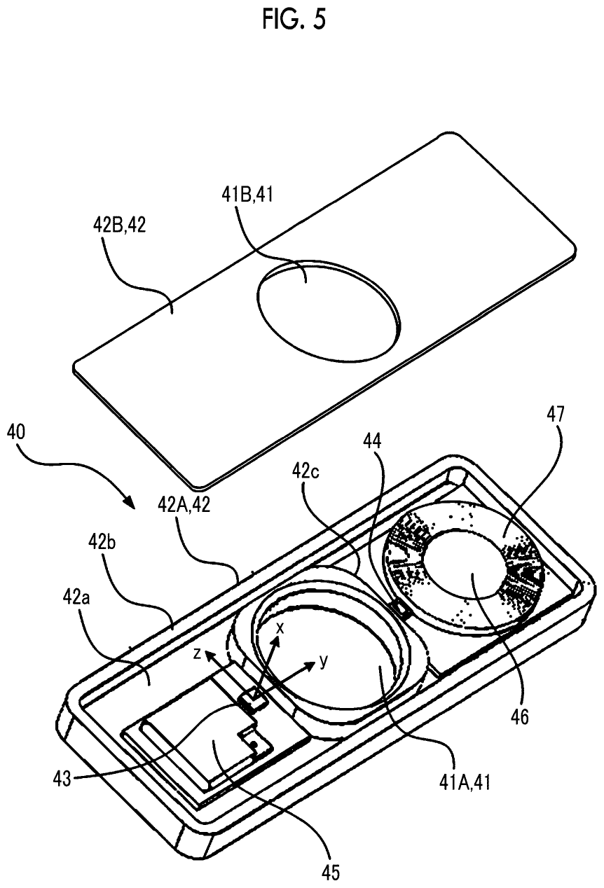

FIG. 5 is an exploded perspective view showing a configuration example of the detection unit 40. The detection unit 40 includes a housing 42 having the through-hole 41, and a magnetic detection unit 43, a magnetic detection unit 44, a communication chip 45, a storage battery 46, and a power receiving coil 47 that are accommodated in the housing 42.

The housing 42 includes a body part 42A including a flat plate portion 42a that has a rectangular flat plate shape and that has a through-hole 41A penetrating in a thickness direction, a side wall portion 42b that has a rectangular frame shape rising from an outer peripheral edge portion of the flat plate portion 42a in the thickness direction of the flat plate portion 42a, and an inner wall portion 42c that has a cylindrical shape rising from a peripheral edge portion of the through-hole 41A in the flat plate portion 42a in the thickness direction of the flat plate portion 42a, and a lid portion 42B that has a rectangular flat plate shape for closing an accommodation space surrounded by the flat plate portion 42a, the side wall portion 42b, and the inner wall portion 42c. The magnetic detection unit 43, the magnetic detection unit 44, the communication chip 45, the storage battery 46, and the power receiving coil 47 are accommodated in this accommodation space.

A through-hole 41B penetrating in the thickness direction is formed on the lid portion 42B, and in a state in which the lid portion 42B closes the accommodation space, the through-hole 41A and the through-hole 41B communicate with each other through an inner peripheral portion of the inner wall portion 42c to form the through-hole 41 into which the endoscope 1 can be inserted. It is preferable that the through-hole 41 has a perfect circular shape as viewed from an axial direction of the inner wall portion 42c (direction in which the endoscope 1 is inserted). The housing 42 is preferably composed of a resin or the like in order to reduce the weight and the cost, and preferably has a structure that prevents moisture from entering the accommodation space.

Each of the magnetic detection unit 43 and the magnetic detection unit 44 is disposed close to the inner wall portion 42c, and is a three-axis magnetic sensor that can detect a magnetic flux density in a direction x (direction along the axis of the through-hole 41) along the axis of the inner wall portion 42c, a magnetic flux density in a radial direction y of the through-hole 41, and a magnetic flux density in a direction z orthogonal to the direction x and the radial direction y.

In a state in which the insertion part 10 of the endoscope 1 is inserted into the through-hole 41, the longitudinal direction X of the insertion part 10 and the direction x match each other, the radial direction Y of the insertion part 10 and the radial direction y match each other, and the circumferential direction Z of the insertion part 10 and the direction z match each other. Therefore, each of the magnetic detection unit 43 and the magnetic detection unit 44 is configured to detect a magnetic flux density BX in the longitudinal direction X of the insertion part 10, a magnetic flux density BY in the radial direction Y of the insertion part 10, and a magnetic flux density BZ in the circumferential direction Z of the insertion part 10. Each of the magnetic detection unit 43 and the magnetic detection unit 44 may include three magnetic sensors, which are a uniaxial magnetic sensor that can detect the magnetic flux density BX, a uniaxial magnetic sensor that can detect the magnetic flux density BY, and a uniaxial magnetic sensor that can detect the magnetic flux density BZ. In the present specification, the magnetic flux density BX constitutes a first magnetic flux density, the magnetic flux density BY constitutes a second magnetic flux density, and the magnetic flux density BZ constitutes a third magnetic flux density.

Each of the magnetic detection unit 43 and the magnetic detection unit 44 need only be able to detect the magnetic flux density including a component in the longitudinal direction X, the magnetic flux density including a component in the radial direction Y, and the magnetic flux density including a component in the circumferential direction Z, and three detection axis directions may not exactly match the longitudinal direction X, the radial direction Y, and the circumferential direction Z, respectively. In the magnetic sensor, in a case in which a first detection axis direction is different from the radial direction Y and the circumferential direction Z, a second detection axis direction is different from the longitudinal direction X and the circumferential direction Z, and a third detection axis direction is different from the radial direction Y and the longitudinal direction X, the magnetic sensor can detect the magnetic flux density including the component in the longitudinal direction X, can detect the magnetic flux density including the component in the radial direction Y, and can detect the magnetic flux density including the component in the circumferential direction Z.

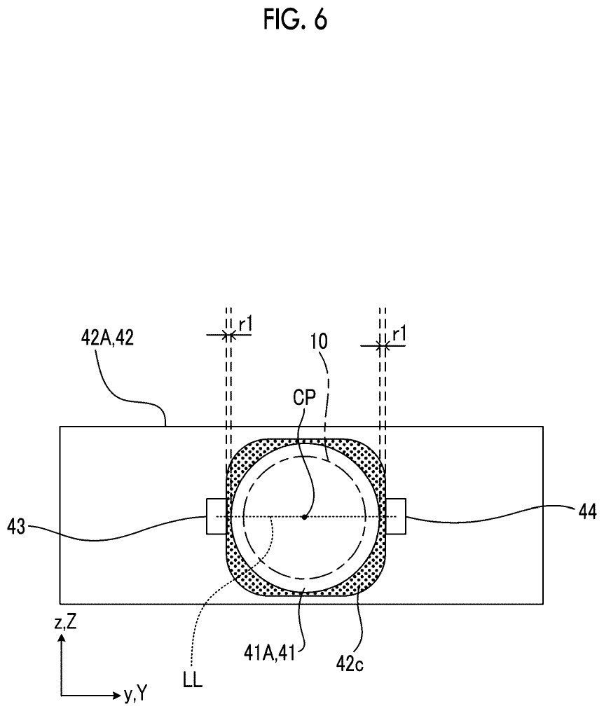

FIG. 6 is a schematic diagram of the body part 42A of the detection unit 40 shown in FIG. 5 as viewed from the direction x. As shown in FIG. 6, the magnetic detection unit 43 and the magnetic detection unit 44 are disposed at positions facing each other with a center CP of the through-hole 41 interposed therebetween as viewed in the direction x. That is, in a state of being viewed in the direction x, a midpoint of a line segment LL connecting the magnetic detection unit 43 and the magnetic detection unit 44 substantially matches the center CP of the through-hole 41. In other words, a distance from the magnetic detection unit 43 to the center CP of the through-hole 41 and a distance from the magnetic detection unit 44 to the center CP of the through-hole 41 substantially match each other.

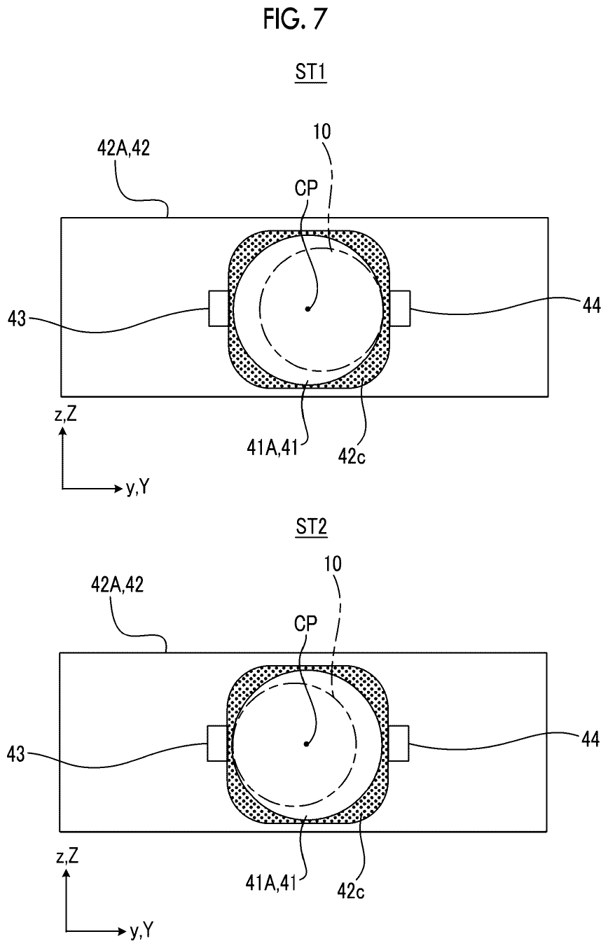

FIG. 7 is a diagram showing an example of a position at which the insertion part 10 can be positioned in the through-hole 41. A state ST1 of FIG. 7 shows a state in which the insertion part 10 is most distant from the magnetic detection unit 43 in the radial direction Y in the through-hole 41. A state ST2 of FIG. 7 shows a state in which the insertion part 10 is most distant from the magnetic detection unit 44 in the radial direction Y in the through-hole 41. A detection range and an installation position of each of the magnetic detection unit 43 and the magnetic detection unit 44 are determined such that the magnetic flux density can be detected with high accuracy from the magnetic pattern formed on the tubular member 17 in any of the state ST1 and the state ST2 of FIG. 7.

In the present embodiment, as shown in FIG. 6, a thickness of a portion of the inner wall portion 42c, the portion being at the same position as the center CP in the direction z, is a thickness r1. The thickness r1 is 0.5 mm, for example. In a case in which the magnetic force of the magnetic pole region formed on the tubular member 17 is defined by the magnetic flux density detected at a position distant from an outer surface of the insertion part 10 in the radial direction of the insertion part 10 by 0.5 mm, it is preferable that the magnetic force has a value that is sufficiently larger than the geomagnetism and is equal to or larger than a value (specifically, 500 microtesla) suitable for the performance of a general magnetic sensor. In addition, for example, in the state ST1 or the state ST2 of FIG. 7, it is more preferable that the magnetic force of the magnetic pole region formed on the tubular member 17 is in a range of 1000 microtesla to 1500 microtesla such that the magnetic detection unit 43 and the magnetic detection unit 44 can detect the magnetic flux density with high accuracy. However, it is preferable that an upper limit value of the magnetic force of the magnetic pole region formed on the tubular member 17 is equal to or less than 20 millitesla such that the insertion part 10 does not adhere to another metal. In consideration of the maximum sensitivity of the general magnetic sensor, it is more preferable that the upper limit value of the magnetic force of the magnetic pole region formed on the tubular member 17 is equal to or less than 2 millitesla.

As shown in FIG. 7, the position of the insertion part 10 in the through-hole 41 may be changed. However, by obtaining the arithmetic mean of the magnetic flux density BX detected from the tubular member 17 by the magnetic detection unit 43 and the magnetic flux density BX detected from the tubular member 17 by the magnetic detection unit 44, it is possible to detect the magnetic flux density BX according to the magnetic pattern regardless of the position of the insertion part 10 in the through-hole 41. Similarly, by obtaining the arithmetic mean of the magnetic flux density BY detected from the tubular member 17 by the magnetic detection unit 43 and the magnetic flux density BY detected from the tubular member 17 by the magnetic detection unit 44, it is possible to detect the magnetic flux density BY according to the magnetic pattern regardless of the position of the insertion part 10 in the through-hole 41. Similarly, by obtaining the arithmetic mean of the magnetic flux density BZ detected from the tubular member 17 by the magnetic detection unit 43 and the magnetic flux density BZ detected from the tubular member 17 by the magnetic detection unit 44, it is possible to detect the magnetic flux density BZ according to the magnetic pattern regardless of the position of the insertion part 10 in the through-hole 41.

The communication chip 45 shown in FIG. 5 transmits information on the magnetic flux density detected by each of the magnetic detection unit 43 and the magnetic detection unit 44 to the expansion device 8 via wireless communication. In the present specification, the communication chip 45 constitutes an output unit that outputs the information detected by the magnetic detection unit 43 and the magnetic detection unit 44 to the outside. This information on the magnetic flux density may be transmitted to the processor device 4, and in this case, this information is transmitted by the processor 4P to the processor 8P of the expansion device 8.

The storage battery 46 is charged by the power received by the power receiving coil 47 by noncontact power supply. The magnetic detection unit 43, the magnetic detection unit 44, and the communication chip 45 are operated by the power supplied from the storage battery 46. The detection unit 40 has a start-up switch (not shown). By performing an operation to turn on the start-up switch, the power supply from the storage battery 46 to the magnetic detection unit 43, the magnetic detection unit 44, and the communication chip 45 is started. The detection unit 40 may have a configuration in which the start-up switch is not provided and the power supply to the magnetic detection unit 43, the magnetic detection unit 44, and the communication chip 45 is started by receiving wireless power supply from the outside. In a case in which the start-up switch is not provided, a structure in which the accommodation space of the housing 42 is completely sealed can be easily realized.

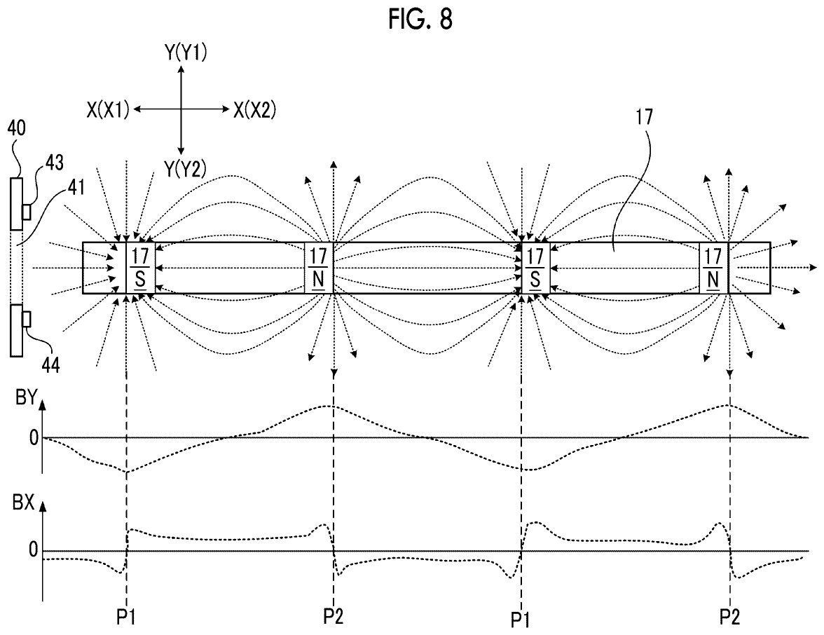

FIG. 8 is a schematic diagram showing an example of the magnetic flux density detected by the magnetic detection unit 43. Since the magnetic flux density detected by the magnetic detection unit 44 is the same as that in FIG. 8, the illustration is omitted. Two graphs shown in FIG. 8 show the magnetic flux density BX and the magnetic flux density BY that are detected by the magnetic detection unit 43 in a case where the soft portion 10A is moved in the longitudinal direction X1 through the through-hole 41. In FIG. 8, a magnetic flux line from the positive pole region 17N to the negative pole region 17S adjacent to the positive pole region 17N in the longitudinal direction X is indicated by a broken line arrow.

In a case in which the soft portion 10A (tubular member 17) is moved toward the through-hole 41 of the detection unit 40 shown in the upper left of FIG. 8, as shown in the graph of FIG. 8, the magnetic flux density BX detected by the magnetic detection unit 43 has a positive value between each positive pole region 17N and the negative pole region 17S adjacent to the positive pole region 17N in the longitudinal direction X1, and has a negative value between each positive pole region 17N and the negative pole region 17S adjacent to the positive pole region 17N in the longitudinal direction X2. In addition, the magnetic flux density BY detected by the magnetic detection unit 43 has a negative value and a large absolute value in the vicinity of the negative pole region 17S, has a positive value and a large absolute value in the vicinity of the positive pole region 17N, and has a value close to zero in the vicinity of an intermediate position between the negative pole region 17S and the positive pole region 17N.

Regarding the magnetic flux densities detected from the magnetic pattern formed on the tubular member 17 at a plurality of positions in the longitudinal direction X of the tubular member 17, each of the magnetic flux density BX and the magnetic flux density BY is periodically changed with positive and negative values, and the phases of the magnetic flux density BX and the magnetic flux density BY are shifted from each other in the longitudinal direction X. In the negative pole region 17S, an end (portion of a position P1 in FIG. 8) in the longitudinal direction X where the absolute value of the magnetic flux density BY is the maximum value is hereinafter referred to as a negative pole end. In the positive pole region 17N, an end (portion of a position P2 in FIG. 8) in the longitudinal direction X where the absolute value of the magnetic flux density BY is the maximum value is hereinafter referred to as a positive pole end.

As an example, by magnetizing the tubular member 17 using the method described above by setting a length of the cylindrical coil of the magnetic field generation device 300 in the axial direction to 60 mm, an inner diameter of the cylindrical coil of the magnetic field generation device 300 to 18 mm, and a movement pitch of the cylindrical coil in the longitudinal direction X to 144 mm, it is possible to form the magnetic pattern in which a distance between the negative pole end and the positive pole end is 72 mm. In the example of FIG. 8, for example, by disposing the cylindrical coil between the negative pole region 17S at the left end and the positive pole region 17N adjacent to the right side of the negative pole region 17S to form the magnetic field, it is possible to form these two magnetic pole regions. Then, from that state, by relatively moving the cylindrical coil by 144 mm in the longitudinal direction X2 to form the magnetic field in that state, it is possible to form the positive pole region 17N at the right end and the negative pole region 17S adjacent to the left side of the positive pole region 17N. In this manner, it is possible to form the magnetic pattern in which the distance (distance between the position P1 and the position P2) between the positive pole end and the negative pole end which are alternately formed in the longitudinal direction X is 72 mm.

In the endoscope system 200, the processor 8P of the expansion device 8 acquires the information on the magnetic flux densities detected by the magnetic detection unit 43 and the magnetic detection unit 44, from the detection unit 40, and determines a movement state of the insertion part 10 in the longitudinal direction X based on the acquired magnetic flux density BX and magnetic flux density BY. The movement state of the insertion part 10 determined here includes a movement direction indicating in which direction in the longitudinal direction X the insertion part 10 is moved with respect to the detection unit 40, and a movement amount (movement distance) indicating how much distance the insertion part 10 inserted into the through-hole 41 of the detection unit 40 has moved in the longitudinal direction X with respect to the detection unit 40. The processor 8P obtains the arithmetic mean of the magnetic flux densities BX respectively detected at the same timing by the magnetic detection unit 43 and the magnetic detection unit 44, obtains the arithmetic mean of the magnetic flux densities BY respectively detected at the same timing by the magnetic detection unit 43 and the magnetic detection unit 44, and determines the movement state of the insertion part 10 based on the magnetic flux density BX and the magnetic flux density BY obtained by the arithmetic mean.

The processor 8P classifies the magnetic flux density BX into a plurality of pieces of information according to the magnitude thereof, classifies the magnetic flux density BY into a plurality of pieces of information according to the magnitude thereof, and determines the movement state of the insertion part 10 in the longitudinal direction X based on a combination of any of the plurality of pieces of information obtained by classifying the magnetic flux density BX and any of the plurality of pieces of information obtained by classifying the magnetic flux density BY.

Specifically, the processor 8P sets a first threshold value th (for example, “0”) as a threshold value for classifying the magnetic flux density BX into two levels, and sets a second threshold value th1 (positive value larger than 0) and a second threshold value th2 (negative value less than 0) as a threshold value for classifying the magnetic flux density BY into three levels. Moreover, the processor 8P classifies the magnetic flux density BX by setting a value larger than the first threshold value th as a high level H and setting a value less than the first threshold value th as a low level L. Further, the processor 8P classifies the magnetic flux density BY by setting a value larger than the second threshold value th1 as the high level H, setting a value between the second threshold value th1 and the second threshold value th2 as a middle level M, and setting a value less than the second threshold value th2 as the low level L. The result of classifying the magnetic flux density BX in this manner is also referred to as a classification level of the magnetic flux density BX, and the result of classifying the magnetic flux density BY in this manner is also referred to as a classification level of the magnetic flux density BY.

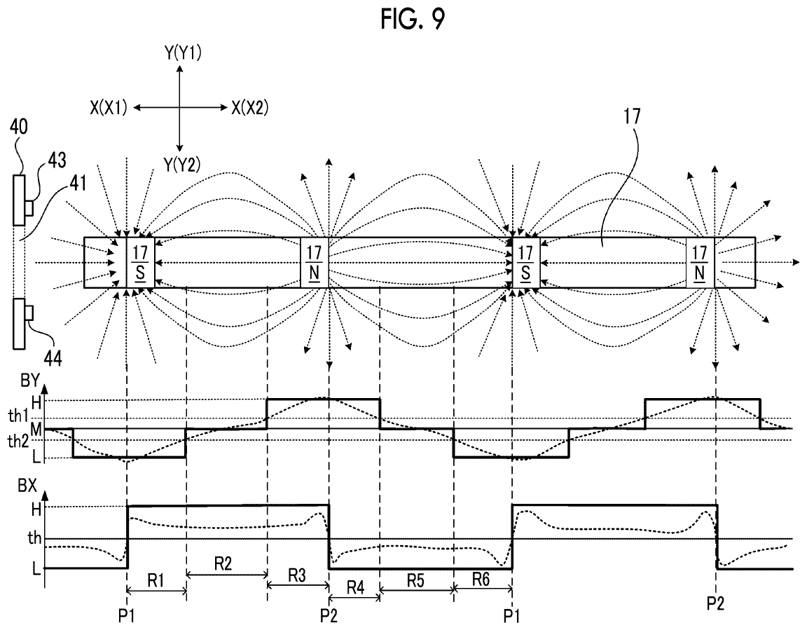

In FIG. 9, the result (classification level) of classifying the magnetic flux density BX and the magnetic flux density BY in the graphs shown in FIG. 8 is indicated by a thick solid line. As shown in FIG. 9, in the tubular member 17, a range between two adjacent positions P1 (between the negative pole ends) is divided into a region R1 in which the magnetic flux density BX is at the high level and the magnetic flux density BY is at the low level; a region R2 in which the magnetic flux density BX is at the high level and the magnetic flux density BY is at the middle level; a region R3 in which the magnetic flux density BX is at the high level and the magnetic flux density BY is at the high level; a region R4 in which the magnetic flux density BX is at the low level and the magnetic flux density BY is at the high level; a region R5 in which the magnetic flux density BX is at the low level and the magnetic flux density BY is at the middle level; and a region R6 in which the magnetic flux density BX is at the low level and the magnetic flux density BY is at the low level. As described above, the range between the negative pole ends adjacent to each other in the longitudinal direction X can be divided into six regions R1 to R6 depending on the combination of the classification level of the magnetic flux density BX and the classification level of the magnetic flux density BY.

By monitoring the thick solid lines (classification levels of the magnetic flux densities BX and BY) shown in FIG. 9, the processor 8P determines the movement direction of the insertion part 10 with respect to the detection unit 40, and the movement amount (movement distance) of the insertion part 10 in the longitudinal direction X starting from the position of the detection unit 40.

For example, in a case in which the negative pole region 17S provided on the most distal end side of the tubular member 17 passes through the through-hole 41, the processor 8P detects that the region R1 at the most distal end of the tubular member 17 is positioned in the through-hole 41, from the combination of the classification level of the magnetic flux density BX and the classification level of the magnetic flux density BY, and detects the position as a reference position. The distance (referred to as a distance L1) in the longitudinal direction X from the negative pole region 17S provided on the most distal end side of the tubular member 17 to the distal end of the distal end part 10C is known. Therefore, in a case in which this reference position is detected, the processor 8P determines that the movement distance of the insertion part 10 with respect to the detection unit 40 is “0”, and further determines that an insertion length (distance from the reference position (through-hole 41) to the distal end of the insertion part 10) of the insertion part 10 into the body of the subject 50 is the distance L1.

After the reference position is detected, in a case in which it is determined according to the classification levels of the magnetic flux densities BX and BY that the region of the tubular member 17 passing through the through-hole 41 is being changed in a direction from the region R1 to the region R6, the processor 8P determines that the insertion part 10 is being moved in the longitudinal direction X1. In addition, in a case in which it is determined that the insertion part 10 is being moved in the longitudinal direction X1, the processor 8P increases the movement distance of the insertion part 10 in the longitudinal direction X1 by a unit distance ΔL and increases the insertion length of the insertion part 10 into the body of the subject 50 by the unit distance ΔL, each time the region of the tubular member 17 passing through the through-hole 41 is changed by one (for example, a change from the region R1 to the region R2 or a change from the region R2 to the region R3). The unit distance ΔL can be a value obtained by dividing an interval between the adjacent negative pole regions 17S by 6.

On the other hand, in a case in which it is determined according to the classification levels of the magnetic flux densities BX and BY that the region of the tubular member 17 passing through the through-hole 41 is being changed in a direction from the region R6 to the region R1, the processor 8P determines that the insertion part 10 is being moved in the longitudinal direction X2. In addition, in a case in which it is determined that the insertion part 10 is being moved in the longitudinal direction X2, the processor 8P decreases the movement distance of the insertion part 10 in the longitudinal direction X1 by the unit distance ΔL and decreases the insertion length of the insertion part 10 into the body of the subject 50 by the unit distance ΔL, each time the region of the tubular member 17 passing through the through-hole 41 is changed by one.

Depending on the movement speed of the insertion part 10, it can also be determined that the region of the tubular member 17 passing through the through-hole 41 is changed from the region R1 to the region R3 or is changed from the region R3 to the region R1. In a case in which it is determined that the region of the tubular member 17 passing through the through-hole 41 is changed by two in this manner, the processor 8P need only increase or decrease the insertion length of the insertion part 10 by twice the unit distance ΔL.

The processor 8P displays the information on the insertion length determined in this manner on the display device 7, outputs the information via voice from a speaker (not shown), or transmits the information to an operator of the endoscope 1 via vibration of a vibrator provided in the operating part 11. As a result, it is possible to accurately record an imaging position via the endoscope 1, guide or evaluate the operation of the endoscope 1, and the like.

As described above, by demagnetizing the distal end part 10C and the bendable part 10B in the insertion part 10, the processor 8P can easily detect the reference position. Specifically, in a case in which the insertion part 10 is inserted into the through-hole 41 from the distal end side and is moved in the longitudinal direction X1, both the magnetic flux density BX and the magnetic flux density BY are values in the vicinity of “0” while the distal end part 10C and the bendable part 10B pass through the through-hole 41. Further, at a point in time at which the negative pole region 17S on the most distal end side of the tubular member 17 reaches the through-hole 41, the magnetic flux density BX and the magnetic flux density BY are a combination of the high level and the low level as shown in FIG. 9, and therefore, it is possible to easily detect the reference position via the fluctuation of the magnetic flux density.

As described above, the processor 8P classifies the magnetic flux density BX into two of the high level and the low level, classifies the magnetic flux density BY into three of the high level, the middle level, and the low level, and determines the movement state of the insertion part 10 in the longitudinal direction X based on the combination thereof. In this way, by monitoring the change in the combination of the classification level of the magnetic flux density BX and the classification level of the magnetic flux density BY, the movement direction, the movement distance, and the insertion length of the insertion part 10 can be determined. With the endoscope system 200, such an effect can be realized only by magnetizing the endoscope 1 having a general-purpose configuration and adding the detection unit 40, so that a construction cost of the system can be reduced. In addition, since the movement direction, the movement distance, and the insertion length of the insertion part 10 are determined based on the information on the magnetic flux density that can be acquired non-optically, even in a case in which the insertion part 10 is dirty, the determination accuracy is not reduced, which is practical.

In addition, by using the combination of the classification level of the magnetic flux density BX and the classification level of the magnetic flux density BY, it is possible to determine the movement distance of the insertion part 10 with a resolution (for example, a unit of ⅓ of an interval) finer than an interval between the two types of adjacent magnetic pole regions (negative pole region 17S and positive pole region 17N). In this way, the movement distance can be finely determined, which can be useful for accurate recording of the imaging position by the endoscope 1, guiding or evaluation of the operation of the endoscope 1, and the like.

In addition, the processor 8P obtains the arithmetic mean of the magnetic flux density detected by the magnetic detection unit 43 and the magnetic flux density detected by the magnetic detection unit 44, and determines the movement direction, the movement distance, and the insertion length of the insertion part 10 based on the magnetic flux density of the arithmetic mean. Therefore, it is possible to obtain the change in the magnetic flux density according to the magnetic pattern regardless of the position of the insertion part 10 in the through-hole 41. In addition, the magnetic flux densities detected by the magnetic detection unit 43 and the magnetic detection unit 44 can include a disturbance component caused by geomagnetism, a magnetic field generated by a steel frame of a building, a magnetic field generated by the steel furniture, and the like, in addition to a magnetic field generated by magnetization. However, as described above, by obtaining the arithmetic mean of the magnetic flux densities detected by the two magnetic detection units, it is possible to reduce an influence of the disturbance component.

In a case in which a difference between an inner diameter of the through-hole 41 and an outer diameter of the insertion part 10 is made as small as possible, any one of the magnetic detection unit 43 or the magnetic detection unit 44 provided in the detection unit 40 is not essential and can be omitted. In this case, the processor 8P need only determine the movement direction, the movement distance, and the insertion length of the insertion part 10 based on the magnetic flux densities BX and BY detected by the magnetic detection unit 43 or the magnetic detection unit 44.

In addition, in the present embodiment, each of the negative pole region 17S and the positive pole region 17N formed on the tubular member 17 is formed in an annular shape along the outer periphery of the tubular member 17. Therefore, even in a case in which the insertion part 10 is rotated in the circumferential direction thereof in the through-hole 41, it is possible to substantially eliminate the change in the magnetic flux densities detected by the magnetic detection unit 43 and the magnetic detection unit 44. Therefore, the movement direction, the movement distance, and the insertion length of the insertion part 10 can be determined regardless of the posture of the insertion part 10.

The disturbance component can be included in the magnetic flux densities detected by the magnetic detection unit 43 and the magnetic detection unit 44. In addition, the orientation of the disturbance component is also changed depending on the posture of the detection unit 40. Therefore, the influence of the disturbance component can be eliminated by classifying the magnetic flux density BX into two of the high level and the low level, classifying the magnetic flux density BY into three of the high level, the middle level, and the low level, and determining the movement state of the insertion part 10 in the longitudinal direction X based on the combination of the classification levels as described above, rather than determining the movement state of the insertion part 10 in the longitudinal direction X using raw data of the magnetic flux density BX and the magnetic flux density BY as they are.

In the above description, the processor 8P classifies the magnetic flux density BX into two of the high level and the low level, classifies the magnetic flux density BY into three of the high level, the middle level, and the low level, and determines the movement state of the insertion part 10 in the longitudinal direction X based on the combination of the classification levels. As a modification example, the processor 8P may classify the magnetic flux density BX into two of the high level and the low level, may classify the magnetic flux density BY into two of the high level and the low level, and may determine the movement state of the insertion part 10 in the longitudinal direction X based on the combination of the classification levels.

Specifically, the processor 8P sets the “first threshold value th (for example, 0)” as the threshold value for classifying the magnetic flux density BX into two levels, and sets a “second threshold value th3 (for example, 0)” as the threshold value for classifying the magnetic flux density BY into two levels. Moreover, the processor 8P classifies the magnetic flux density BX by setting a value larger than the first threshold value th as the high level and setting a value less than the first threshold value th as the low level. Further, the processor 8P classifies the magnetic flux density BY by setting a value larger than the second threshold value th3 as the high level and setting a value less than the second threshold value th3 as the low level.

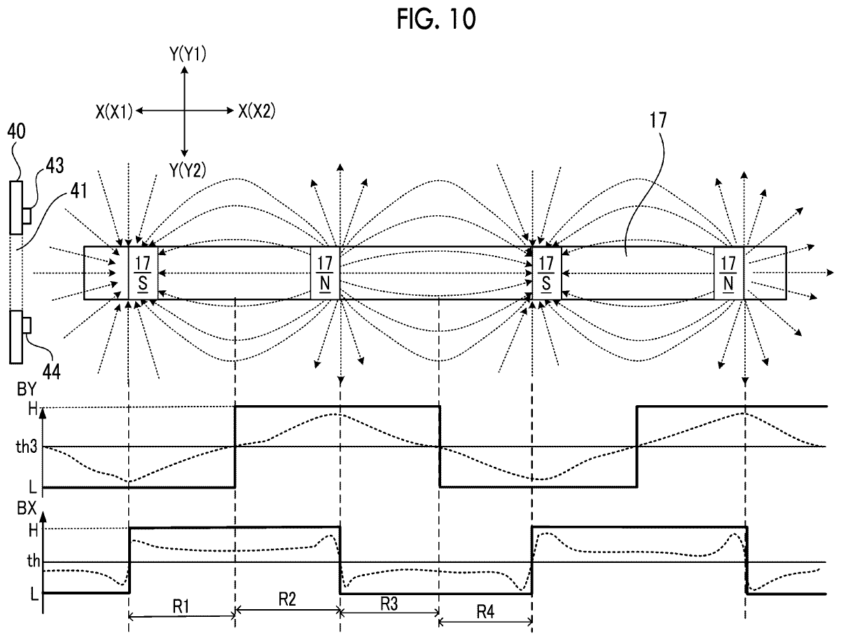

In FIG. 10, the result (classification level) of classifying the magnetic flux density BX and the magnetic flux density BY in the graphs shown in FIG. 8 is indicated by a thick solid line. As shown in FIG. 10, in the tubular member 17, a range between two adjacent positions P1 is divided into a region R1 in which the magnetic flux density BX is at the high level and the magnetic flux density BY is at the low level, a region R2 in which the magnetic flux density BX is at the high level and the magnetic flux density BY is at the high level, a region R3 in which the magnetic flux density BX is at the low level and the magnetic flux density BY is at the high level, and a region R4 in which the magnetic flux density BX is at the low level and the magnetic flux density BY is at the low level. As described above, the range between the negative pole ends adjacent to each other in the longitudinal direction X can be divided into four regions R1 to R4 depending on the combination of the classification level of the magnetic flux density BX and the classification level of the magnetic flux density BY. By monitoring the thick solid lines (classification levels of the magnetic flux densities BX and BY) shown in FIG. 10, the processor 8P can determine the movement direction of the insertion part 10 and the movement amount (movement distance) of the insertion part 10 in the longitudinal direction X.

In the description above, the processor 8P classifies the magnetic flux density into the plurality of pieces of information according to the magnitude thereof. However, a configuration may be adopted in which this classification is performed by a processor provided in the communication chip of the detection unit 40. That is, a configuration may be adopted in which the detection unit 40 transmits information on the classification level indicated by the thick solid line shown in FIG. 9 or FIG. 10 to the processor 8P. In addition, the processor 8P performs the determination of the movement state of the insertion part 10, but a configuration may be adopted in which the processor provided in the communication chip of the detection unit 40 performs the determination to transmit the determination result to the processor 8P. Further, a configuration may be adopted in which a processor such as a personal computer connected to the expansion device 8 via a network acquires the information on the magnetic flux density from the detection unit 40, performs the determination, and transmits the determination result to the processor 8P. Also, a processor separate from the processor 8P may perform the determination of the movement state of the insertion part 10. Further, a configuration may be adopted in which a processor provided outside the endoscope device 100 performs the determination of the movement state of the insertion part 10 to transmit the determination result to the processor 8P.

The threshold value used in a case of classifying each of the magnetic flux density BX and the magnetic flux density BY according to the magnitude thereof may be a predetermined fixed value, but the threshold value is preferably a variable value to be determined based on the magnetic flux densities detected by the magnetic detection unit 43 and the magnetic detection unit 44 after the insertion of the insertion part 10 into the through-hole 41 is started.

For example, in a case in which the start-up switch of the detection unit 40 is turned on, the insertion part 10 is inserted into the through-hole 41, and the third magnetic pole region from the most distal end side of the tubular member 17 passes through the through-hole 41, the processor 8P can acquire each of the maximum value and the minimum value of the magnetic flux density BX detected by the magnetic detection unit 43, and the maximum value and the minimum value of the magnetic flux density BY detected by the magnetic detection unit 43. In a case where the maximum value and the minimum value of the magnetic flux density BX are acquired, the processor 8P obtains an average value of the maximum value and the minimum value, and sets the average value as the first threshold value th. Further, in a case in which the maximum value and the minimum value of the magnetic flux density BY are acquired, the processor 8P obtains an average value of the maximum value and the minimum value, sets a value obtained by adding a predetermined value to the average value as the second threshold value th1, and sets a value obtained by subtracting a predetermined value from the average value as the second threshold value th2. The predetermined value is a value that is larger than a value assumed as the disturbance component and is less than the absolute value of each of the maximum value and the minimum value of the magnetic flux density BY. The first to third magnetic pole regions from the most distal end side of the tubular member 17 constitute a proximal end part on the demagnetized region (adjacent region) side in the region in which the magnetic pattern is formed.

Hereinafter, the processor 8P need only classify the magnetic flux density BX and the magnetic flux density BY by using the threshold values set in this manner. In this way, it is possible to perform the determination of the movement state of the insertion part 10 with higher accuracy by setting the threshold values based on the magnetic flux densities detected by the magnetic detection unit 43 and the magnetic detection unit 44.

In this way, in a case in which the threshold value is set based on the magnetic flux densities detected by the magnetic detection unit 43 and the magnetic detection unit 44, it is preferable that, in a period until the third magnetic pole region from the most distal end side of the tubular member 17 passes through the through-hole 41, the processor 8P sets the first threshold value th, the second threshold value th1, and the second threshold value th2 to the predetermined values, performs the detection of the reference position and the determination of the movement state of the insertion part 10, and then updates the first threshold value th, the second threshold value th1, and the second threshold value th2 via the method described above to perform the determination of the movement state of the insertion part 10.

As described above, in the endoscope system 200, the magnetic pattern is formed on the tubular member 17 such that the magnetic flux densities BX and BY detected by each of the magnetic detection unit 43 and the magnetic detection unit 44 are changed periodically between positive and negative and phases thereof are shifted in a case in which the insertion part 10 passes through the through-hole 41, so that it is possible to perform the determination of the movement state of the insertion part 10. Such a magnetic pattern is not limited to the configurations of the magnetic pole portions MA1 and MA2 shown in FIGS. 3 and 4, and can be variously modified.

### Processing of Processor 8P

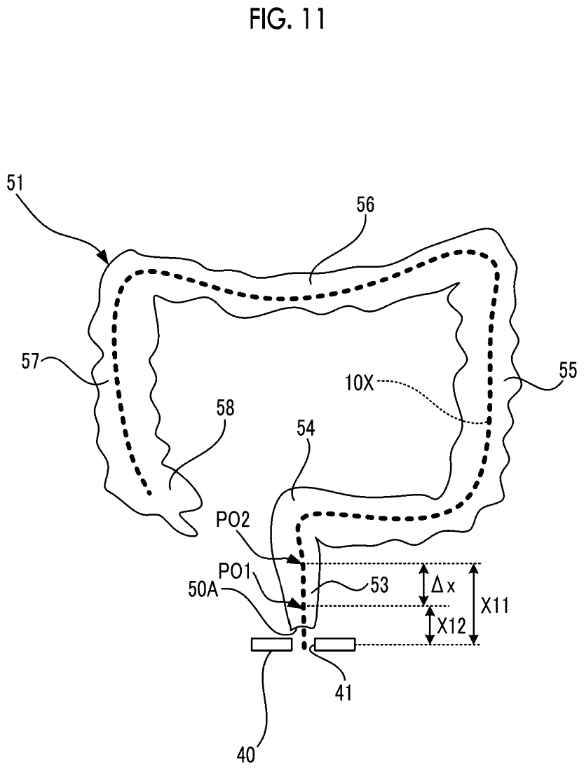

Next, details of various kinds of processing executed by the processor 8P will be described. In order to describe these various kinds of processing, the movement path of the insertion part 10 of the endoscope 1 will be described. FIG. 11 is a schematic diagram showing the movement path of the insertion part 10 in an examination (hereinafter, referred to as endoscopy) performed using the endoscope 1.

The endoscopy includes an endoscopy that examines an upper digestive organ such as a stomach and an endoscopy that examines a lower digestive organ such as a large intestine. In addition, the endoscopy includes a first examination in which the insertion part 10 is inserted into the subject in order to examine whether or not a lesion region is present in the subject, and a second examination in which the insertion part 10 is inserted into the subject in order to excise an already known lesion region.

### Movement Path of Endoscope

FIG. 11 shows a large intestine 51 of the subject (subject 50). In the endoscopy of the large intestine, the insertion part 10 is moved along a movement path 10X indicated by a broken line in the drawing. The movement path 10X is a tubular path from the through-hole 41 of the detection unit 40 disposed in the vicinity of the anus 50A outside the subject through the anus 50A to a rectum 53, and further from the rectum 53 through a sigmoid colon 54, a descending colon 55, a transverse colon 56, and an ascending colon 57 to an ileocecum 58.

### Processing During Endoscopy

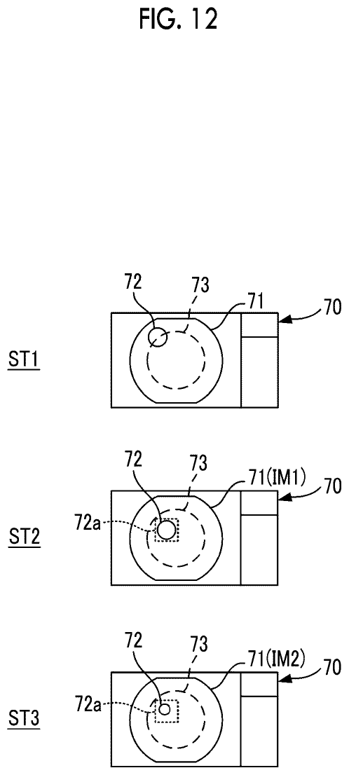

Hereinafter, the operator of the endoscope 1 will be simply referred to as an operator. FIG. 12 is a diagram showing a transition example of a screen 70 displayed on the display device 7 during endoscopy. In a case where the endoscopy is started, the power of the detection unit 40 is turned on. As described above, the processor 8P derives a first distance (hereinafter, referred to as insertion length) from the reference position (position of the through-hole 41) on the movement path 10X to the distal end of the insertion part 10 based on the magnetic flux densities BX and BY detected by the detection unit 40. The operator moves the endoscope 1 along the movement path 10X while checking the captured image data 71 of the endoscope 1 on the display device 7 to search for the lesion candidate region.

For example, in a state in which the distal end of the endoscope 1 is at the position PO2 shown in FIG. 11, in a case in which the lesion candidate region 72 is imaged, the operator operates a switch or the like included in the operating part 11 to give an instruction to recognize the lesion candidate region. The processor 8P that has received this instruction acquires the captured image data 71 captured by the endoscope 1 from the processor device 4 and performs the lesion recognition processing on the captured image data 71. In addition, the processor 8P displays a guide frame 73 for guiding the position of the lesion candidate region on the display device 7 (State ST1 in FIG. 12). The operator moves the endoscope 1 such that the lesion candidate region 72 is positioned inside the guide frame 73. It is preferable that the guide frame 73 is set in a range with less distortion in the captured image data (imaging range in which a distortion amount is equal to or smaller than the threshold value).

As a result of the lesion recognition processing, the processor 8P recognizes that the lesion candidate region 72 is included in the captured image data 71, and further, in a case where it is determined that the lesion candidate region 72 is positioned inside the guide frame 73, the processor 8P acquires the insertion length X11 when the captured image data 71 is captured while being displayed, and records the insertion length X11 in the memory in association with the captured image data 71. In addition, the processor 8P displays a frame 72a (for example, a rectangular frame that includes the lesion candidate region 72) indicating the contour of the lesion candidate region 72 on the display device 7 (a state ST2 in FIG. 12). The insertion length X11 constitutes information related to the imaging position of the captured image data 71 in the state ST2.

Next, the operator moves the endoscope 1 in the longitudinal direction X2. In this case, the operator moves the endoscope 1 such that a state in which the lesion candidate region 72 is positioned inside the frame 72a is maintained. With this movement, the screen 70 is changed to a state ST3. Even during the movement of the endoscope 1, the processor 8P acquires the insertion length, and in a case where the difference between the insertion length X11 recorded in the memory and the newly acquired insertion length X12 is equal to or larger than the threshold value and the lesion candidate region 72 is positioned within the frame 72a, records the insertion length X12 in the memory in association with the captured image data 71 obtained in that state. The insertion length X12 constitutes information related to the imaging position of the captured image data 71 in the state ST3. In the example in FIG. 11, the difference between the insertion length X12 and the insertion length X11 is equal to or larger than the threshold value in a state in which the distal end of the endoscope 1 is at the position PO1 (corresponding to a state ST3 in FIG. 12).

The captured image data 71 (captured image data obtained at the position PO2) in the state ST2 and the captured image data 71 (captured image data obtained at the position PO1) in the state ST3 include the same lesion candidate region 72, but are obtained by imaging at different timings. Here, in order to distinguish between the two, the captured image data 71 in the state ST2 is also referred to as first medical image data IM1, and the captured image data 71 in the state ST3 is also referred to as second medical image data IM2.

Next, the processor 8P derives the actual size of the lesion candidate region 72 based on the first medical image data IM1, the insertion length X11 that is information related to the imaging position of the first medical image data IM1, the second medical image data IM2, and the insertion length X12 that is information related to the imaging position of the second medical image data IM2. The size of the lesion candidate region refers to a maximum length in a direction perpendicular to the optical axis of the endoscope 1 or a plane area in a case of being viewed in the direction perpendicular to the optical axis.

For example, the processor 8P acquires the image size Y11 of the lesion candidate region 72 in the first medical image data IM1 based on the first medical image data IM1 and records the acquired image size Y11 in the memory. Further, the processor 8P acquires the image size Y12 of the lesion candidate region 72 in the second medical image data IM2 based on the second medical image data IM2 and records the acquired image size Y12 in the memory. The image size of the lesion candidate region in the captured image data is specifically indicated by the number of pixels, and is a larger one of the number of pixels in the horizontal direction and the number of pixels in the vertical direction. The image size of the lesion candidate region in the captured image data may be acquired as an area value obtained by multiplying the total number of pixels constituting the lesion candidate region by a predetermined area per pixel.

Since the image size Yₙ is smaller as the distance Xₙ is larger, a relationship of the distance Xₙ from the distal end of the endoscope 1 to the lesion candidate region, the actual size S of the lesion candidate region, and the image size Yₙ in the captured image data of the lesion candidate region can be expressed by an equation of Yₙ=α×S/X, as a predetermined constant α. Therefore, the actual size S can be derived by using the above equation with two different distances Xₙ and image sizes Yₙ.

For example, two equations having different distances Xₙ and image sizes Yₙ are represented by the following equations (A) and (B).

$${Y}_{1} = α \times S / {X}_{1} \qquad ( A )$$

$${Y}_{2} = α \times S / {X}_{2} \qquad ( B )$$

Equation (A) can be modified to Equation (C), and Equation (B) can be modified to Equation (D).

$${X}_{1} = α \times S / {Y}_{1} \qquad ( C )$$

$${X}_{2} = α \times S / {Y}_{2} \qquad ( D )$$

In a case where |X₁−X₂|=Δx, the following Equation (E) is obtained from Equation (C) and Equation (D).

$$Δ x = ( α \times S \times \vert {Y}_{2} - {Y}_{1} \vert ) / ( {Y}_{1} \times {Y}_{2} ) \qquad ( E )$$

From Equation (E), the actual size S can be derived by Equation (F).

$$S = ( {Y}_{1} \times {Y}_{2} \times Δ x ) / ( α \times \vert {Y}_{2} - {Y}_{1} \vert ) \qquad ( F )$$

Δx in Equation (F) corresponds to a difference between the insertion length X11 and the insertion length X12, Y₁ corresponds to the image size Y11, and Y₂ corresponds to the image size Y12. Therefore, the processor 8P can derive the actual size S of the lesion candidate region 72 by substituting Δx obtained from the insertion length X11 and the insertion length X12, the image size Y11, and the image size Y12 into Equation (F).

The constant α in Equation (F) may be changed for each type (model) of the endoscope 1. Therefore, it is preferable that the constant α is recorded for each type of the endoscope 1, and the processor 8P recognizes the type of the connected endoscope 1, selects and uses the constant α corresponding to the recognized type, and derives the actual size S by the calculation of Equation (F). As a result, the actual size S can be derived with high accuracy. The constant α may also change for each individual endoscope 1. Therefore, even in a case where the endoscope 1 of the same type is used, it is preferable to actually measure and record the constant α for each individual endoscope 1.

Further, the constant α may be changed depending on the position of the lesion candidate region 72 in the captured image data. For example, the constant α may be changed between a case in which the lesion candidate region 72 is positioned close to the outer peripheral edge of the guide frame 73 and a case in which the lesion candidate region 72 is positioned close to the center of the guide frame 73. Therefore, it is preferable that a plurality of values of the constant α are recorded for each position (distance from the center of the captured image data) of the lesion candidate region 72 in the captured image data, and the processor 8P selects the constant α in accordance with the position of the lesion candidate region 72 to derive the actual size S of the lesion candidate region 72. As a result, the actual size S can be derived with high accuracy. As described above, the guide frame 73 is set in the region with less distortion in the captured image data, so that the derivation accuracy of the actual size S can be improved.

In the present embodiment, in a case where the first medical image data IM1 is acquired, the frame 72a indicating the contour of the lesion candidate region 72 is displayed on the display device 7, and the captured image data 71 acquired in a state in which the lesion candidate region 72 is present in the frame 72a is used as the second medical image data IM2. As a result, the distance (distance on the image) between the position of the lesion candidate region in the first medical image data IM1 and the position of the lesion candidate region in the second medical image data IM2 can be set to the threshold value or less. Since the positions of the lesion candidate regions 72 in the two captured image data are close to each other, the values of a in Equation (A) and Equation (B) can be treated as the same, and thus the actual size S can be derived with high accuracy.

The processor 8P derives the actual size S by the calculation using Equation (F), but the present disclosure is not limited thereto. For example, a data table in which the above-described combination of the insertion length X11, the insertion length X12, the image size Y11, and the image size Y12 and the actual size S as numerical data are associated with each other is created and recorded in the memory of the processor 8P. Moreover, in a case in which the insertion length X11, the insertion length X12, the image size Y11, and the image size Y12 are acquired, the processor 8P may read out and derive the actual size S corresponding to a combination of the insertion length X11, the insertion length X12, the image size Y11, and the image size Y12 from the data table. The data table may be generated by actually measuring an object having a known size imaged by the endoscope 1.

As described above, by using the data table, the derivation of the actual size S can be performed at a higher speed. In addition, since the calculation is not necessary, the circuit of the processor 8P can be simplified, which leads to a reduction in manufacturing cost. It is preferable that the data table is recorded for each type of the endoscope 1, and the data table corresponding to the type of the endoscope 1 is selected to derive the actual size S. In addition, it is preferable that the data table is recorded for each position of the lesion candidate region, and the data table corresponding to the position of the lesion candidate region is selected to derive the actual size S.

As described above, according to the endoscope system 200, the actual size of the lesion candidate region can be derived without using the auxiliary measurement light or the like, and it is possible to prevent an increase in manufacturing cost. The processor 8P acquires two captured image data (first medical image data IM1 and second medical image data IM2 in which a distance between two imaging positions of the first medical image data IM1 and the second medical image data IM2 is equal to or larger than a threshold value) in which the difference between the insertion length X11 and the insertion length X12 is equal to or larger than the threshold value, and derives the actual size S based on the acquired two captured image data. Therefore, it is possible to derive the actual size S with high accuracy by eliminating the influence of the detection resolution or the detection error of the insertion length.

In a case where a plurality of lesion candidate regions 72 are included in each of the first medical image data IM1 and the second medical image data IM2, it is preferable that the processor 8P sets a lesion candidate region 72 having a relatively large (preferably, maximum) image size among the plurality of lesion candidate regions 72 as a target for derivation of the actual size. For example, in a case in which the actual size of the lesion candidate region 72 having the maximum image size is known, the operator can also estimate the approximate actual size of the lesion candidate region 72 having an actual size smaller than the maximum image size.

As described above, the detection unit 40 can also be integrally configured with an insertion assisting member of the endoscope 1. For example, the detection unit 40 may be integrally formed with the insertion assisting member to be inserted into the anus, or may be integrally formed with a mouthpiece-type insertion assisting member that is held in the mouth. In addition, the detection unit 40 may be integrally formed with pants for endoscopy, or may be configured to be attachable to and detachable from the pants for endoscopy.

The technique of the present disclosure is not limited to the above description, and can be appropriately changed as described below.

For example, the endoscope 1 may be inserted into the body through the mouth or a nose of the subject 50. In this case, the detection unit 40 need only have a shape to be attachable to the mouth or the nose of the subject 50.

The tubular member 17 has the configuration in which the first member 14 and the second member 15 are provided, and each of the first member 14 and the second member 15 contains the magnetizable austenitic stainless steel, but one of the first member 14 or the second member 15 may be made of a non-magnetizable material. That is, the magnetic pattern may not be formed on one of the first member 14 or the second member 15. Even in such a case, since the magnetic flux densities BX, BY, and BZ described above can be detected from the tubular member 17, it is possible to determine the movement state of the insertion part 10.

In the above description, in the tubular member 17, the two types of magnetic polo regions are alternately arranged in the longitudinal direction to form the magnetic pattern, and the movement state of the insertion part 10 in the longitudinal direction is determined based on the combination of the classification levels of the magnetic information in the two directions detected from the magnetic pattern. However, the two types of magnetic pole regions formed on the tubular member 17 may not be alternately arranged in the longitudinal direction. Even in such a case, the movement state of the insertion part 10 in the longitudinal direction can be determined based on the combination of the classification levels of the magnetic information in the two directions detected from the magnetic pattern.

In addition, as a modification example, the movement state of the insertion part 10 in the longitudinal direction may be determined by forming a pattern more complicated than the magnetic pattern on the tubular member 17 and detecting the pattern via the magnetic detection units 43 and 44. Specifically, a table in which each position of the tubular member 17 in the longitudinal direction and the magnetic flux density BX or the magnetic flux density BY (classification level) detected at each position are associated with each other may be recorded in a memory, and the processor 8P may classify the magnetic flux density BX or the magnetic flux density BY detected by the magnetic detection unit 43 to acquire the classification level, and may acquire the information on the position corresponding to the classification level from the table to determine the insertion length of the insertion part 10. As a result, the insertion length of the insertion part 10 can be finely determined. In addition, the magnetic detection units 43 and 44 can detect the magnetic flux densities in one direction, so that the cost can be reduced.

Instead of providing the magnetic pattern as described above, the endoscope 1 may be provided with a motion sensor such as an acceleration sensor in the distal end part. In this case, the Δx may be derived based on output information of the acceleration sensor in a case where the first medical image data IM1 is acquired and output information of the acceleration sensor in a case where the second medical image data IM2 is acquired.

In the above description, although the endoscope is described as an example of the medical image acquisition device, the technique of the present disclosure can be applied to any device capable of acquiring the medical image data including the lesion candidate region at different positions. For example, the technique of the present disclosure can also be applied to an ultrasound diagnostic apparatus capable of acquiring ultrasound image data as medical image data.

As described above, at least the following matters are described in the present specification. In the following, the components corresponding to the above-described embodiments are shown in parentheses, but the present disclosure is not limited thereto.

(1)

A processing device (expansion device 8) comprising:
a processor (processor 8P),
in which the processor is configured to:
   acquire first medical image data (captured image data 71 in state ST2) and second medical image data (captured image data 71 in state ST3) including a specific region (lesion candidate region 72) in a subject as medical image data acquired by a medical image acquisition device (endoscope 1);
   acquire first information (insertion length X11) regarding an imaging position of the first medical image data and second information (insertion length X12) regarding an imaging position of the second medical image data; and
   derive an actual size of the specific region based on the first medical image data, the second medical image data, the first information, and the second information.
      (2)

The processing device according to (1),
in which the processor may be configured to:
   derive a first size (image size Y11) of the specific region in the first medical image data;
   derive a second size (image size Y12) of the specific region in the second medical image data; and
   derive the actual size based on the first size, the second size, the first information, and the second information.
      (3)

The processing device according to (2),
in which the medical image acquisition device may be an endoscope.
   (4)

The processing device according to (3),
in which the processor may be configured to further derive the actual size based on positions of the specific regions in the first medical image data and the second medical image data.
   (5)

The processing device according to (4),
in which the processor may be configured to derive the actual size by performing calculation using the first size, the second size, the first information, the second information, and a predetermined constant (constant α).
   (6)

The processing device according to (5),
in which the processor may be configured to change the constant based on the position of the specific region.
   (7)

The processing device according to any one of (3) to (6),
in which the processor may be configured to acquire the first medical image data and the second medical image data in which a distance between a position of the specific region in the first medical image data and a position of the specific region in the second medical image data is in a relationship of being equal to or less than a threshold value.
   (8)

The processing device according to any one of (3) to (7),
in which the processor may be configured to further derive the actual size based on a type of the endoscope.
   (9)

The processing device according to (3),
in which the processor may be configured to acquire numerical data corresponding to a combination of the first size, the second size, the first information, and the second information from a predetermined data table, and derive the numerical data as the actual size.
   (10)
   The processing device according to (9),
in which the processor may be configured to change the data table based on a type of the endoscope.
   (11)

The processing device according to (9) or (10),
in which the processor may be configured to change the data table based on positions of the specific regions in the first medical image data and the second medical image data.
   (12)

The processing device according to any one of (3) to (11),
in which a magnetic pattern may be formed in an insertion part of the endoscope, and the processor may be configured to acquire the first information and the second information based on a magnetic field detected by a detection unit (magnetic detection unit 43 and magnetic detection unit 44) disposed outside a subject into which the endoscope is inserted from the magnetic pattern.
   (13)

The processing device according to (12),
in which the processor may be configured to acquire the first medical image data and the second medical image data in which a distance between the imaging position of the first medical image data and the imaging position of the second medical image data is in a relationship of being equal to or greater than a threshold value.
   (14)

The processing device according to any one of (3) to (13),
in which the processor may be configured to derive, in a case where a plurality of the specific regions are included in the first medical image data and the second medical image data, a size of one region which has a relatively large size among the specific regions as the first size and the second size.
   (15)

An endoscope device (endoscope device 100) comprising:
the processing device according to any one of (3) to (13); and
the endoscope.
   (16)

The endoscope device according to (15), further comprising:
a detection unit (magnetic detection unit 43 and magnetic detection unit 44) that is disposed on a movement path of the endoscope,
   in which an insertion part of the endoscope includes a member (tubular member 17) that contains metal in which a magnetic pattern is integrally formed,
   the detection unit detects a magnetic field from the member, and
   the processor is configured to acquire the first information and the second information based on the magnetic field detected by the detection unit.
      (17)

The endoscope device according to (16),
in which the insertion part may include a soft portion of the endoscope.
   (18)

The endoscope device according to (17),
in which the soft portion has a cylindrical member (outer skin layer 18) having an insulating property, a cylindrical first member (first member 14) that contains metal and that is covered with the cylindrical member, and a cylindrical second member (second member 15) that contains metal and that is inserted into the first member, and the member includes at least one of the first member or the second member.
   (19)

The endoscope device according to (18),
in which the at least one of the first member or the second member may be made of magnetizable austenitic stainless steel.
   (20)

A processing method comprising:
acquiring first medical image data and second medical image data including a specific region in a subject as medical image data acquired by a medical image acquisition device;
acquiring first information regarding an imaging position of the first medical image data and second information regarding an imaging position of the second medical image data; and
deriving an actual size of the specific region based on the first medical image data, the second medical image data, the first information, and the second information.
   (21)

A processing program causing a processor to execute:
a step of acquiring first medical image data and second medical image data including a specific region in a subject as medical image data acquired by a medical image acquisition device;
a step of acquiring first information regarding an imaging position of the first medical image data and second information regarding an imaging position of the second medical image data; and
a step of deriving an actual size of the specific region based on the first medical image data, the second medical image data, the first information, and the second information.

### EXPLANATION OF REFERENCES

1: endoscope
MA, MA1, MA2: magnetic pole portion
4P: processor
4: processor device
5: light source device
6: input unit
7: display device
8: expansion device
8P: processor
10A: soft portion
10B: bendable part
10C: distal end part
10: insertion part
11: operating part
12: angle knob
13A, 13B: connector portion
13: universal cord
14: first member
15a: metal strip
15: second member
16A, 16B: cap
17N: positive pole region
17S: negative pole region
17: tubular member
18A: inner resin layer
18B: outer resin layer
18: outer skin layer
19: coating layer
40: detection unit
42: housing
42A: body part
42B: lid portion
42a: flat plate portion
42b: side wall portion
42c: inner wall portion
41A, 41B, 41: through-hole
43, 44: magnetic detection unit
45: communication chip
46: storage battery
47: power receiving coil
50A: anus
53: rectum
54: sigmoid colon
55: descending colon
56: transverse colon
57: ascending colon
58: ileocecum
50: subject
100: endoscope device
200: endoscope system
300: magnetic field generation device
PO1, PO2: position

What is claimed is:

1. A processing device comprising:
a processor,
wherein the processor is configured to:
acquire first medical image data and second medical image data that include a specific region in a subject as medical image data acquired by a medical image acquisition device;
acquire first information regarding an imaging position of the first medical image data and second information regarding an imaging position of the second medical image data; and
derive an actual size of the specific region based on the first medical image data, the second medical image data, the first information, and the second information.

2. The processing device according to claim 1,
wherein the processor is configured to:
derive a first size of the specific region in the first medical image data;
derive a second size of the specific region in the second medical image data; and
derive the actual size based on the first size, the second size, the first information, and the second information.

3. The processing device according to claim 2,
wherein the medical image acquisition device is an endoscope.

4. The processing device according to claim 3,
wherein the processor is configured to further derive the actual size based on positions of the specific regions in the first medical image data and the second medical image data.

5. The processing device according to claim 4,
wherein the processor is configured to derive the actual size by performing calculation using the first size, the second size, the first information, the second information, and a predetermined constant.

6. The processing device according to claim 5,
wherein the processor is configured to change the predetermined constant based on the positions of the specific regions.

7. The processing device according to claim 3,
wherein the processor is configured to acquire the first medical image data and the second medical image data, in which a distance between a position of the specific region in the first medical image data and a position of the specific region in the second medical image data is equal to or less than a threshold value.

8. The processing device according to claim 3,
wherein the processor is configured to further derive the actual size based on a type of the endoscope.

9. The processing device according to claim 3,
wherein the processor is configured to acquire numerical data corresponding to a combination of the first size, the second size, the first information, and the second information from a predetermined data table, and derive the numerical data as the actual size.

10. The processing device according to claim 9,
wherein the processor is configured to change the data table based on a type of the endoscope.

11. The processing device according to claim 9,
wherein the processor is configured to change the data table based on positions of the specific regions in the first medical image data and the second medical image data.

12. The processing device according to claim 3,
wherein a magnetic pattern is formed in an insertion part of the endoscope, and
the processor is configured to acquire the first information and the second information based on a magnetic field detected by a detection circuit disposed outside the subject into which the endoscope is inserted from the magnetic pattern.

13. The processing device according to claim 12,
wherein the processor is configured to acquire the first medical image data and the second medical image data, in which a distance between the imaging position of the first medical image data and the imaging position of the second medical image data is equal to or greater than a threshold value.

14. The processing device according to claim 3,
wherein the processor is configured to derive, in a case where a plurality of the specific regions are included in the first medical image data and the second medical image data, a size of one region which has a relatively large size among the specific regions as the first size and the second size.

15. An endoscope device comprising:
the processing device according to claim 3; and
the endoscope.

16. The endoscope device according to claim 15, further comprising:
a detection circuit that is disposed on a movement path of the endoscope,
wherein an insertion part of the endoscope includes a member that contains metal in which a magnetic pattern is integrally formed,
the detection circuit detects a magnetic field from the member, and
the processor is configured to acquire the first information and the second information based on the magnetic field detected by the detection circuit.

17. The endoscope device according to claim 16,
wherein the insertion part includes a soft portion of the endoscope.

18. The endoscope device according to claim 17,
wherein the soft portion has a cylindrical member having an insulating property, a first member that contains metal and that is covered with the cylindrical member, and a second member that contains metal and that is inserted into the first member, the first member and the second member being cylindrical, and
the member includes at least one of the first member or the second member.

19. The endoscope device according to claim 18,
wherein the at least one of the first member or the second member includes magnetizable austenitic stainless steel.

20. A processing method comprising:
acquiring first medical image data and second medical image data that include a specific region in a subject as medical image data acquired by a medical image acquisition device;
acquiring first information regarding an imaging position of the first medical image data and second information regarding an imaging position of the second medical image data; and
deriving an actual size of the specific region based on the first medical image data, the second medical image data, the first information, and the second information.

21. A non-transitory computer-readable storage medium storing a processing program causing a processor to execute a process, the process comprising:
acquiring first medical image data and second medical image data that include a specific region in a subject as medical image data acquired by a medical image acquisition device;
acquiring first information regarding an imaging position of the first medical image data and second information regarding an imaging position of the second medical image data; and
deriving an actual size of the specific region based on the first medical image data, the second medical image data, the first information, and the second information.
